(19) United States
(12) Patent Application Publication
(10) Pub. No.: US 2018/0120325 A1
(43) Pub. Date: May 3, 2018

(54) Sequence Specific Fluorescence for Peptide-Fluorochrome Interactions

(71) Applicant: MedChem Imaging, Boston, MA (US)

(72) Inventor: Lee Josephson, Reading, MA (US)

(73) Assignee: MedChem Imaging, Boston, MA (US)

(21) Appl. No.: 15/728,502

(22) Filed: Oct. 10, 2017

Related U.S. Application Data
(60) Provisional application No. 62/405,798, filed on Oct. 7, 2016.

(51) Int. Cl. G01N 33/68; G01N 21/64

(52) U.S. Cl. CPC G01N 33/6803; G01N 2333/47; G01N 2021/6439; G01N 21/6428

(57) ABSTRACT

A method of obtaining fluorochromes that bind to specific sites on proteins is presented. The method consists of synthesizing a peptide bearing a sequence from a parent protein and a second peptide of similar composition but with a scrambled sequence. Each peptide is then added to a test fluorochrome at similar concentrations, and a difference in fluorescence is obtained. A difference in fluorescence indicates a sequence dependent interaction between the peptide and fluorochrome. A large number of fluorochromes can be screened using the two peptides to find one binding in a sequence dependent fashion. This fluorochrome can then be tested for binding to the original full-length protein. The fluorochrome can then serve as scaffold in medicinal chemistry. The fluorochrome/peptide interaction and resulting fluorescence can be inhibited by non-fluorescent test compounds, which are compounds that bind to this peptide. Thus. the peptide/fluorochrome interaction can provide assays screening for therapeutic drugs binding to this site on the protein.

### CROSS REFERENCE TO RELATED APPLICATION

This application is a continuation of U.S. Application Ser. No. 61/405,798, entitled, “Sequence Specific Fluorescence for Peptide-Fluorochrome Interactions,” filed Oct. 7, 2016, the entire disclosure of which is incorporated herein by reference.

### FIELD OF THE INVENTION

The invention provides materials and methods of using peptide/fluorochrome interactions between a fluorochrome and a peptide.

Throughout this application, various publications are referenced to within parentheses. Disclosures of these publications in their entireties are hereby incorporated by reference into this application to more fully describe the state of the art to which this invention pertains. Full bibliographic citations for these references may be found at the end of this application, preceding the claims.

### BACKGROUND OF THE INVENTION

The deposition of proteins as amyloid fibrils is characteristic of many neurodegenerative diseases (“NDDs”), and many other diseases termed “Systemic Amyloidoses.” Among the neurodegenerative diseases are Alzheimer's disease, where amyloid fibrils are composed of the ABeta amyloid peptide; Parkinson's Disease, where the fibrils are composed of alphasynuclein protein (“ASyn”); Huntington's Disease, where the fibrils are exon 1 of the Huntingtin protein;, and tauopathies, e.g. Alzheimer's disease, and frontotemporal dementia, having various forms of tau) (1).

Among the Systemic Amyloidoses are type 2 diabetes, with fibrils of islet amyloid polypeptide (“IAPP,” “amylin”); and familial amyloidosis, with fibrils of the transerythrin protein. Compilations of amyloids and amyloid associated diseases are available (2). Hereafter, I shall refer to amyloid forming biomolecules (peptides, polypeptides and proteins) as “proteins,” regardless of amino acid chain length.

A beta sheet backbone is a common structural element of the amyloid fibrils of diverse amyloid-forming proteins. Amyloid fibril beta sheet(s) (“BS”) are formed when two linear amino acid chains bind each other through hydrogen bonds between the nitrogen hydrogen (N—H) and carbonyl oxygen (C═O) of the peptide backbone. Beta sheets can be parallel (two N-termini and two C-termini at each end) or antiparallel (one N-termini and one C-termini at each end). See (3, 4). In many amyloids, the linear Beta sheet sequences of amino acids are connected by sharp bends termed “hairpins,” though a hairpin is not a necessary condition of Beta Sheet formation. The hairpin beta sheet (“HBS's”) structures of the ABeta protein amyloids are shown in FIG. 1.

The design of diagnostic drugs for imaging amyloid currently is based on low molecular weight (<2000 Da) amyloid-binding, microscopy stains like Thioflavin T, Congo Red or Chysamine G. These stains serve as leads for the design structurally related compounds that can be radiolabeled, and which have physical properties for needed for drug use, including blood brain barrier penetration. Fluorescent or birefringent stains bind in crevices formed by the amino acid side chains of Beta Sheets or hairpin beta sheets, see FIG. 1A. For compilations of such probes and the imaging agents designed from them, see (5-7).

Assays for therapeutic drugs inhibiting amyloid formation often use Beta Sheet binding probes that fluoresce when binding. For example, the effects of test compounds on amyloid formation can be monitored by the increase in fluorescent stain that Thioflavin T (ThT) undergoes when it binds amyloid Beta Sheets (8). Assay protocols using ThT fluorescence or Congo Red birefringence to measure amyloid formation are available at http://www.assay-protocol.com/, along with kits employing this principle (SensoLyte® Amyloid Aggregation Kit from Anaspec). Radiolabeled Beta Sheet-binding probes are used in conjunction with a separation of bound and free, as in radioligand assays to assess the strength of amyloid binding (9).

However, since the Beta Sheet or Hairpin Beta Sheet structure is common to diverse amyloids and other non-amyloid forming proteins, diagnostic or therapeutic drugs targeted to them have limited selectivity. For example, the PET imaging agent [¹¹C]-PIB developed to image ABeta amyloid of Alzheimer's disease, binds a variety of amyloids (10) and can be used to image ATTR amyloid (11). The need to develop an amyloid specific PET or SPECT imaging agent for ASyn, an amyloid protein associated with Parkinson's disease (PD), has been widely recognized. See (12, 13) and the Michael J. Fox Foundation (MJFF) website. (MJFF is a leading research organization dedicated to curing Parkinson's disease.) A high concentration of ABeta peptide in tissues with ASyn makes the development of an ASyn imaging agent with necessary selectively for ASyn extremely challenging. Similarly, therapeutic agents binding Beta Sheet targets will also have limited molecular specificity, since the common Beta Sheet structure is found in both amyloid and non-amyloid forming proteins.

There is a need for methods for measuring the interactions of molecules with non-Hairpin Binding Sheet (“nHBS”) regions of amyloid forming proteins that can be used in the development amyloid specific imaging agents or amyloid targeted therapeutic drugs. The invention uses fluorescence measurements of sequence specific fluorochrome/peptide interactions to achieve this goal.

### SUMMARY OF THE INVENTION

The invention uses changes in emitted fluorescent to determine sequence specific interactions between a fluorochrome and a peptide. A conformationally restricted peptide bearing a known sequence of longer peptide or protein and a scrambled version of the same sequence are selected and separately assessed for interaction with a fluorochrome. A difference in emission of fluorescence of the peptide over the scrambled peptide is indicative of an interaction between the peptide and the fluorochrome.

In one aspect of the invention, the difference in fluorescence emission is an increase in emission of the peptide fluorochrome interaction over the scrambled peptide fluorochrome interaction. In one aspect of the invention, the difference in fluorescence emission is a decrease in emission of the peptide fluorochrome interaction over the scrambled peptide fluorochrome interaction.

In other aspects of the invention, the peptide bears a known sequence to a non-hairpin binding sheet of an amyloid forming protein. In other aspects, the non-hairpin binding beta-sheet protein is a portion of the protein Asyn. In other aspects, the non-hairpin binding beta sheet protein is a portion of the protein tau.

In another aspect of the invention, FRET can be used to detect changes in fluorescence.

In another aspect of the invention, the invention can be used to detect interactions between a non-fluorescent molecule and a peptide or protein. A non-fluorescent compound is allowed to interact with a peptide-fluorochrome pair and the emitted fluorescence is measured. A decrease in fluorescence over the emitted fluorescence of the peptide fluorochrome pair is indicative of an interaction of the compound with the peptide.

### BRIEF DESCRIPTION OF THE DRAWINGS

FIG. 1A shows that the amyloid fibrils of the ABeta peptide are composed of ABeta Sheet pleated sheet structures. The ABeta peptides form “hairpin loops” and fibrils. Thioflavin T and similar dyes bind in a crevasse along fibril axis. (FIG. 1A & 1B are from (A) (14), (B) (26), (C) (27), (D) & (E) (28)). The fluorochrome thioflavin T (ThT), a small molecule reporter, binds in crevices formed by amino acid side chains of the ABeta beta sheets formed by multiple ABeta peptides.

FIG. 1B represents the time course of a typical small molecule reporter fluorescent assay for the amyloid cascade used to screen for inhibitors of the amyloid cascade. During the lag phase, small aggregates are formed, which do not bind Thioflavin T, and no fluorescence is observed. During the elongation phase, fluorescence increases, reflecting the presence of increasing amounts of beta sheet.

FIG. 2 shows amino acid sequences of five amyloid forming proteins, ABeta, ASyn, Huntingtin, IAPP and Tau. Hairpin Beta Sheet or Beta Sheet sequences are in italics with un-italicized letters indicating amino acids that are not involved in Beta Sheet formation. Examples of charged, conformationally restrained amino acid sequences occurring within the non-Hairpin Beta Sheet or non-Beta Sheet regions of the protein are indicated by underlining.

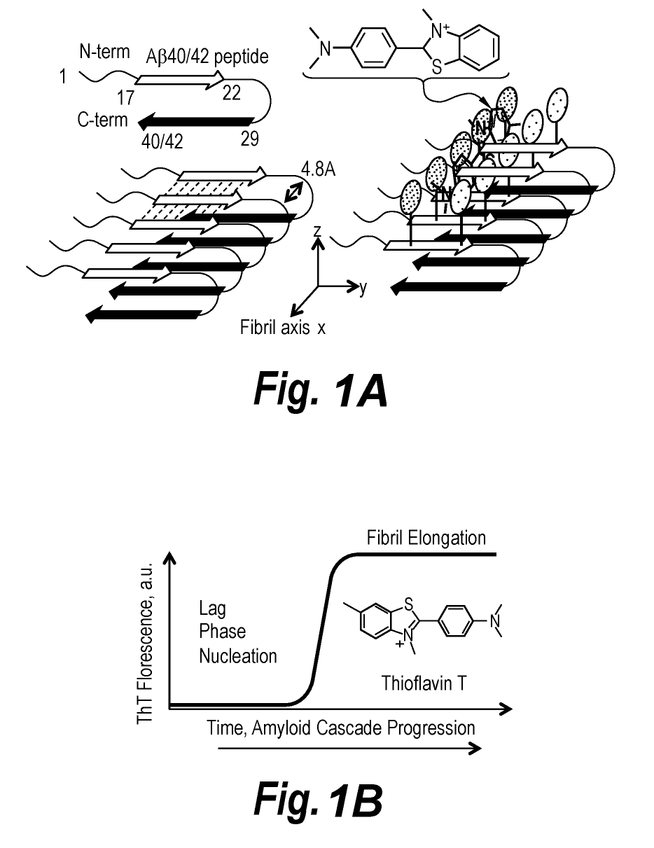

### TERMS AND DEFINITIONS

Amyloid: aggregates of proteins often found as fibrillary structures associated with diverse diseases termed amyloidoses.

Beta Sheet: A secondary structure found in many proteins, and the backbone of the amyloid fibril. The hydrogen atoms of peptide nitrogen atoms form hydrogen bonds with the oxygen atoms of peptide carbonyls. Beta Sheets can be parallel (comprised of two peptides with N-termini an C-termini at the same end) or anti-parallel (one N-terminus and C-terminus at each end.)

Hairpin Beta Sheet: A Beta Sheet where a sharp turn or hairpin occurs between sequences forming a beta sheet.

Conformationally restricted sequence: a sequence of amino acids whose conformation in solution is restricted by a mechanisms such as its amino acid content (e.g. high proline density), or by disulfide bonds between cysteine groups, salt bridges between positive to negatively charged amino acid side chains, or covalent linkages, either natural or created in vitro.

Fluorochrome: A molecule of less than about 1000 Da that absorbs light between roughly 300 and 1000 nm and emits light at a lower energy and longer wavelength.

Ligand: a substance that forms a complex with a biomolecule like a peptide.

Peptide(nHBS): a peptide bearing a sequence form an amyloid forming protein that is not involved in Beta Sheet (BS) or Hairpin Beta Sheet (HBS) formation.

Scrambled peptide: a peptide of the same length, same molecular weight and same amino acid composition as a reference peptide, but bearing no sequence similarity to the reference peptide, identified as Peptide(nHBS)_{scrm}.

Sequence specific interaction with a peptide: An interaction, such as that measured by fluorescence, that is stronger with a peptide bearing a sequence of a protein, denoted Peptide(nHBS), and weaker with a scrambled version of the same peptide, denoted Peptide(nHBS)_{scrm}.

Fluor(nHBS): a fluorochrome which interacts with Peptide(nHBS) in a sequence specific fashion.

All other terms not defined in this specification are given their usual and customary definitions as used by one of ordinary skill in the field.

Specific preferred embodiments of the invention will become evident from the following more detailed description of certain preferred embodiments and the claims.

### DETAILED DESCRIPTION OF THE INVENTION

The invention uses changes in emitted fluorescent to determine sequence specific interactions between a fluorochrome and a peptide. A conformationally restricted peptide bearing a known sequence of longer peptide or protein and a scrambled version of the same sequence are selected and separately assessed for interaction with a fluorochrome. A difference in emission of fluorescence of the peptide over the scrambled peptide is indicative of an interaction between the peptide and the fluorochrome.

The invention presents methods of measuring fluorescence that yields sequence specific fluorochrome/peptide probes that bind to peptides, proteins, and to non-Beta Sheet portions of amyloid forming proteins. The invention then applies the peptide fluorochrome binding principle to determine ability of test compounds to serve as diagnostic drugs or therapeutic drugs. The fluorescence measurements can be employed to competitive, homogeneous assay formats. In one aspect, the invention can detect interaction with non-hairpin beta sheet peptide sequences of amyloid forming proteins with fluorochromes, and then the ability of test compounds to interact with that portion of the amyloid protein.

One aspect of the invention consists of the following steps.

1. Determine amino acid sequence(s) of the nHBS region(s) from within the full sequences of amyloid forming proteins, and synthesize a non-FIBS peptide (Peptide(nHBS)) and control-scrambled peptide (Peptide(nHBS_{scram}).

2. Obtain a panel of fluorochromes.

3. Measure the differential fluorescence arising from the addition of Peptide(nHBS) and Peptide(nHBS-scram) to each fluorochrome from (II). Select a fluorochrome (denoted “Fluoro(nHBS)”) with a large differential in fluorescence upon the addition of the two peptides.

Amyloid Forming Proteins

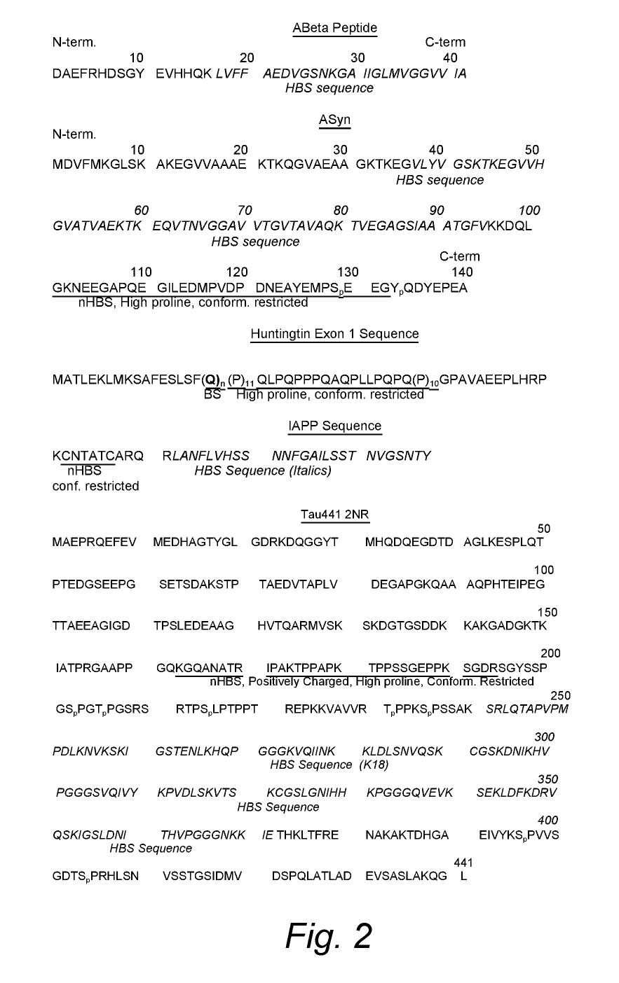

Amyloid-forming proteins are comprised of sequences of amino acids, some of which are involved in the Beta Sheet or Hairpin Beta Sheet, and some of which are not (“non-Beta Sheet” or “non-Hairpin Beta Sheet”). FIG. 2 indicates regions involved in Hairpin Beta Sheet structures with italics, with non-Hairpin Beta Sheet sequences un-italicized, for five amyloid forming proteins: ABeta (14), Huntingtin exon 1 (15), ASyn (16), islet-forming amyloid polypeptide (IAPP or amylin) (17, 18), and the 441 amino acid long version of tau (19).

As shown in FIG. 2, Huntingtin Exon 1 is a sequence from the far longer Huntingtin protein, which forms the amyloid in Huntington's disease. Exon 1 forms amyloid due to mutations producing extended sequences of glutamine (poly Q regions). Normal persons have Huntingin with polyQ's of less than 35 amino acids, while individuals affected with Huntington's disease have far longer polyQ's (20, 21).

ASyn, the amyloid in Parkinson's disease, has five linear Beta Sheet sequences and four hairpins comprising the Hairpin Beta Sheet region (16). The non-Hairpin Beta Sheet N-terminal residues of ASyn (1-36) have a high sequence similarity to beta and gamma synucleins, which makes them unusable for the development of ASyn selective fluorochromes or imaging agents (22). However the C-terminal region (approximately amino acids 100-140) is unique to ASyn. Thus it is used in example 1, where a peptide bearing the underlined sequence from this region of ASyn is used.

In FIG. 2, the IAPP associated with type 2 diabetes has its Beta Sheet-forming region indicated by italics. A disulfide cyclized series of amino acids near the N-terminus restricts conformation flexibility (17).

For tau, the Hairpin Beta Sheet portion of the long 441 amino acid comprises the K18 region of the paired helical fragment (PHF) core that consists of residues 244 to 372. (23). A positively charged, proline rich, non-Hairpin Beta Sheet region of Tau (underlined) is shown in FIG. 2 and used in Example 3.

A preferred method of obtaining non-Hairpin Beta Sheet peptides is to select those peptides with (i) local regions of conformational restriction and (ii) with significant net negative or net positive charges when synthesized. Conformational restriction is indicated by a high proline content, a disulfide bond, or the presence of in vivo post-translational modifications. The latter indicate these sequences serve as substrates for enzymatic modification in vivo, for example by serine/threonine kinases or tyrosine kinases. Net charge assists peptide solubility and provides a basis for selecting fluorochromes, which can use in part electrostatic forces to bind the nHBS derived peptide.

The importance of non-Hairpin Beta Sheet regions in controlling amyloid formation makes non-Hairpin Beta Sheet regions of amyloid forming proteins potential targets in therapeutic drug development. Although non-Hairpin Beta Sheet sequences are structurally distinct from the Hairpin Beta Sheet, the non-Hairpin Beta Sheet portions of amyloid forming proteins play an important role controlling amyloid formation and presumably in disease development. For example, ASyn mutations associated with the hereditary forms of Parkinson's disease often occur outside the Hairpin Beta Sheet region of amino acids 37 to 95 (24). With ASyn, long range, tertiary interactions allow the C-terminal region (approximately 100 to 140) to stabilize soluble forms and reduce aggregation (25). Since amyloid formation is altered by the non-Hairpin Beta Sheet regions of amyloid forming proteins, fluorescence measurements using the sequence specific interactions of non-Hairpin Beta Sheet peptides and fluorochromes can be used for therapeutic drug development.

Overview of Methods For Practicing the Invention

Fluorescence measurements of the interactions between a fluorochrome (Fluor) and Peptide(nHBS) can be used in homogeneous fluorescent assays, where libraries of compounds, including compounds that are possible therapeutics, are screened for inhibiting the Fluor/Peptide(nHBS) interactions, and reducing the fluorescence resulting from the binding of these two compounds. For example, using a homogeneous, competitive assay format, test compounds can be added a mixture of Fluor and Peptide(nHBS), and the ability to inhibit fluorescence used to infer the binding of test compounds to Peptide(nHBS). In another embodiment, the non-Hairpin Beta Sheet peptides can be synthesized with a fluorochrome covalently attached to obtain fluorescent resonance energy transfer (FRET). Various compounds can be added to inhibit FRET.

The Fluor(nHBS) can serve as “leads,” providing a structural scaffold for the design of chemically related compounds serving as starting points for the development of libraries of compounds designed to serve ultimately diagnostic or therapeutic drugs. Probes used to detect amyloid fibrils by fluorescence or birefringence (Table 1 of (3)) have served as scaffolds for the design of radioactive imaging agents (6). These can be designed to pass through the blood brain barrier by reducing charge and optimizing properties like cLogP and LogP.

An overview of the methods that yield fluorescent probes that bind the non-Beta Sheet portions of amyloid forming proteins follows:

I. Design and synthesis of a non-Hairpin Binding Sheet peptide and a control-scrambled peptide denoted nHBS_{scrm}. The amino sequence of an amyloid forming protein is obtained and location of the Binding Sheet or non-Hairpin Binding Sheet region of the protein determined. FIG. 2 gives the amino acid sequences of four amyloid forming proteins: ABeta protein, Alphasynuclein (ASyn), islet amyloid polypeptide peptide (IAPP, amylin), Huntingtin, and Tau. The Hairpin Binding Sheet involved amino acids are shown in italics, the remainder being the non-Hairpin Binding Sheet sequence.

In one embodiment a non-Hairpin Binding Sheet peptide is selected to be conformationaly restricted. Conformationally restricted sequences can be proline rich (e.g. C-terminus of ASyn), comprised of consecutive proline residues (e.g. polyQ of Huntingtin), or employ disulfide bonds to form cyclic peptide structures (IAPP).

Non-Hairpin Binding Sheet peptides can also be selected for sequence uniqueness. For example, peptide taken from amino acids 1-35 of ASyn have sequences that are shared by other synucleins, while those take from 95 to 140 do not (29).

Non-Hairpin Binding Sheet peptides can be selected to have an overall net negative or net positive charge, which assists in peptide solubility and provides some guidance for selecting those fluorochromes, which though electrostatic interactions are likely to bind the nHBS peptide.

Finally one can synthesize a series of non-Hairpin Binding Sheet coded peptides scanning the entire nHBS region and test each for interaction with a fluorochrome. When an interaction is obtained as an increase in the fluorescence, the corresponding scrambled peptide can be synthesized and the process repeated.

Based an analysis of the Hairpin Binding Sheet and non-Hairpin Binding Sheet regions of the protein, a non-Hairpin Binding Sheet peptide (denoted Peptide(nHBS)), and a scrambled version of this peptide is designed (denoted Peptide(nHBS)_{scrm}), are then designed. Peptide(nHBS) is from the non-BS portion of the protein while Peptide(nHBS)_{scrm} has the same amino acid composition and molecular weight as Peptide(nHBS) but a completely different amino acid sequence. FIG. 2 show the complete sequences of four amyloid proteins, the nHBS region in italics, the some preferred and conformationally restricted regions within the nHBS portion which can be used to obtain Peptide(nHBS) for ASyn, Huntingtin and Tau.

II. Assemble a panel of fluorochromes: Test panels of fluorochromes can be assembled or synthesized using literature procedures. Table 1 shows a panel of positively charged fluorochromes from commercial sources for evaluation for sequence-specific interactions with a negatively charged, ASyn C-terminal peptide. Examples of the syntheses of fluorochrome panels in the literature include those based on bodipy (30, 31), thiazole (32-34), zwitterions (35, 36), and cyanine (37-39).

III. Determine sequence specific peptide induced fluorescence: Increasing concentrations of Peptide(nHBS) and Peptide(nHBS)_{scrm} are added to each fluorochrome from (II) and through fluorescence measurements the best fluorochrome, denoted Fluor(HBS) is selected.

The invention is further illustrated by the following examples, so that a person of ordinary skill in the art may obtain better understanding of the invention. These examples are only provided as exemplary to illustrate the invention, and should not interpreted to limit the invention.

### EXAMPLES

### Example 1

### Method of Determining Sequence Specific Fluorochrome-Peptide Interactions Between a Fluorochrome and a Non-Hairpin Beta Sheet Peptide from ASyn.

Peptide(nHBS) is (NAc)GKNEEGAPQEGILEDMPVDPDNEAYEMPSEEG-NH2 (SEQ. ID No. 1), while Peptide(nHBS)_{scrm} is (NAc)PGPNEKEVAEMQIDLEGDYDNEAEGSEMEPGP-NH2 (Seq. ID. No. 2). Peptide(nHBS) is amino acids 100-132 of human ASyn (FIG. 2) and consists of 10 negative amino acids (aspartate (D), and glutamate (E)) and one positive amino acid (lysine (K), so it is highly negatively charged. Peptide(nHBS) and Peptide(nHBS)_{scrm} have identical amino acid compositions and negative charges but different sequences.

Each peptide (100 uL in PBS, at various concentrations) will be added to a test fluorochrome from a panel of test fluorochromes (100 uL in PBS) to a 96 well microtiter plate suitable determination of fluorescence using a microtiter plate reader. The concentration of test fluorochrome is between 1 and 1000 nM, preferably between about 5 and 50 nM. A panel of test fluorochromes is shown in Table 1. Peptide concentrations will be varied from about that of the fluorochrome concentration to about 100 times greater than that of the fluorochrome concentration. The two solutions are mixed and allowed to incubate at room temperature for between 15 and 150 minutes, preferably for about 30 minutes. To assess the fluorescence of each well, fluorescence microtiter plate readers such as the Tecan Spark 10M or Biotek Flx800 will be employed.

A sequence dependent fluorochrome/peptide interaction is indicated when Peptide(nHBS) produces a greater fluorescence change than Peptide(nHBS)_{scrm}. Data can be analyzed using the usual 4-parameter equation (parameters of maximum fluorescence, minimum fluorescence, EC50 and n or slope) using Priszm software. The fluorochrome producing the lowest EC50 for the change in fluorescence and a change of fluorescence of at least more 2-fold is selected as the nHBS binding lead fluorochrome and is denoted Fluor(nHBS). Fluor(nHBS) is now known to bind to Peptide(nHBS) in a sequence dependent fashion selected. Fluor(nHBS) must be shown to (i) not bind ABeta amyloid and (ii) bind the full length ASyn protein.

A demonstration of the selectivity of Fluor(nHBS) for binding Asyn and not Beta Sheets (such as ABeta amyloid) is shown by the inability of Fluor(nHBS) to displace radioactive tracers like ¹²⁵I-IMPY, which bind to ABeta amyloid immobilized on filter paper.

To demonstrate Fluoro(nHBS) binds the full length ASyn protein. Some or all of the following experiments are run and yield the following results:

1. Addition of ASyn protein to Fluoro(nHBS) in the microtiter plate format above produces an increase in fluorescence.

2. Monoclonal antibodies which bind to an epitope within the nHBS (e.g. LB509 which binds to amino acids 115 to 122 of human ASyn), are added to the microtiter plate format above and decrease fluorescence.

3. A radioactive version of Fluoro(HBS) binds to ASyn-bearing tissue sections by autoradiography and the distribution of radioactivity is parallel to that seen with anti-ASyn antibodies. The radioactive version of Fluoro(HBS) is obtained by tritium exchange, and tritium autoradiography is employed.

### Example 2

### Method of Determining Sequence Specific Fluorochrome-Peptide Interactions Between a Fluorochrome and a Non Hairpin Beta Sheet Peptide from Tau.

The amino acids 163 to 195 of Tau441R are nHBS, are proline rich, and have a net positive charge, see FIG. 2. In this case, the Peptide(nHBS) is KGQANATRIPAKTPPAPKTPPSSGEPPKSGDR (Seq. ID. No. 3). The peptide has six positively charged lysine groups (K) plus arginines (R) and one negatively charged glutamate (E), for net positive charge. A scrambled nHBS_{scr} peptide is also synthesized. A negatively charged panel of commercially available, negatively charged fluorochromes is used. The procedure from Example 1 is followed to select a Fluor(nHBS) that shows greater fluorescence with Peptide(nHBS) than Peptide(nHBS)_{scrm}. Fluorescence measurements performed as in Example 1 are followed to determine fluorochrome binding to the Peptide(nHBS) in a sequence dependent fashion.

Since this peptide is positively charged, a panel of negatively charged fluorochromes is employed, such as the AlexaFluor carboxylic acids obtained by hydrolysis of AlexaFluor N-Hydroxyl-succinimide (NETS) esters. See Table 2. Purchased AlexaFluor N-Hydroxyl-succinimide esters can be reacted with ethanolamine of ethylenediamine to reduce their negative charges over that obtained with the free carboxyl group used in NHS Ester formation.

### Example 3

### Method of Determining Sequence Specific Fluorochrome-Peptide Interaction by Fluorescence Resonance Energy Transfer (FRET), Indicating

Fluorescence measurements using a fluorescence resonance energy transfer (FRET) donor/acceptor pair are employed to determine sequence dependent Fluoro(nHBS)/Peptide(nHBS) interactions, and their inhibition by a test compound. FRET occurs when there is spectral overlap between the emission spectrum of one fluorochrome and excitation spectrum of a second fluorochrome, and when the two fluorochromes are close to each other, generally less than about 10 nm. For FRET there are two fluorochromes, one of which (e.g. a donor) is covalently attached both to the peptide and to the scrambled peptide). A test fluorochrome (an acceptor) can bind to the peptide (in a sequence dependent fashion). When such binding occurs, energy transfer can occur between the two fluorochromes. If energy transfer does not occur with donor-fluorochrome-scrambled peptide, the interaction is sequence specific.

A wide range of amino reactive Alex Fluors with different excitation and emission maxima are available from vendors like Thermo-Fisher, see Table 2. These can be attached to the N-terminus as the final step of solid phase synthesis. Alternatively, Alex Fluoro NHS esters can be attached to a reactive N-terminus with a solution phase reaction. To effect a selective reaction with N-terminus, the sole lysine needs to be replaced with positively charged, unreactive amino acid like arginine.

A Fluor(nHBS)/Peptide(nHBS) pair is selected as from Examples 1 and 2, and a suitable donor fluorochrome is attached to Peptide(nHBS). For example the NHS ester of an Alex Fluor is attached to the N-terminus of Peptide(nHBS), yielding a FRET donor peptide (AlexFluo)GKNEEGAPQEGILEDMPVDPDNEAYEMPSEEG-NH2 (Seq. ID. No. 4).

### Example 4

### Method of Determining Interactions of Non-Fluorescent Test Compounds with an nHBS Peptide.

To a mixture of Peptide(nHBS) and Fluor(nHBS) is added 10 uL of a 10 mM solution of a test compound in DMSO (or diluted to lower than 10 mM with DMSO). Fluorescence measurements indicate an interaction between the test compound and Peptide(nHBS) when the addition of a test compound causes a reduction in fluorescence.

TABLE 1
Fluorescent Dyes

| Structure | Name | ex | em |
|---|---|---|---|
| 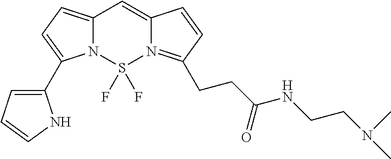 | Lysotracker Red DND-99 | 577 | 590 |
| 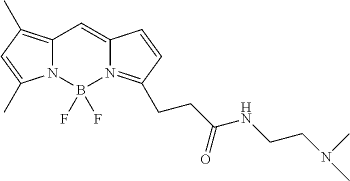 | Lysotracker Green DND-26 | 504 | 511 |
| 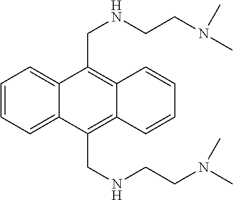 [SMILES: CN(C)CCNCc1c2ccccc2c(CNCCN(C)C)c2ccccc12] | Lysotracker DND- 22 | 373 | 422 |
| 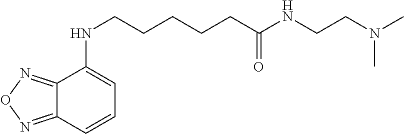 [SMILES: CN(C)CCNC(=O)CCCCCNc1cccc2nonc12] | Lysotracker Yellow HSK-123 | 465 | 535 |
| Lysotracker Yellow HSK-123 | | | |
| 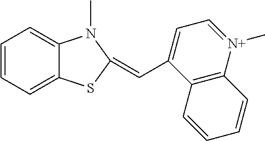 [SMILES: CN1/C(=C\c2cc[n+](C)c3ccccc23)Sc2ccccc21] | Thiazole Orange | 510 | 530 |
| Thiazole Orange (TO) | | | |
| 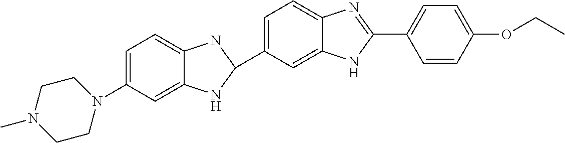 [SMILES: CCOc1ccc(-c2nc3ccc(C4Nc5ccc(N6CCN(C)CC6)cc5N4)cc3[nH]2)cc1] | Hoechst 33342 | 343 | 483 |
| Hoechst 33342 | | | |
| 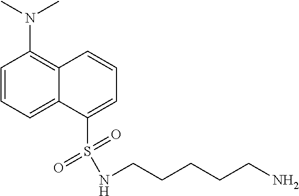 [SMILES: CN(C)c1cccc2c(S(=O)(=O)NCCCCCN)cccc12] | Monodansyl Cadaverine MDC | 338 | 500 |
| 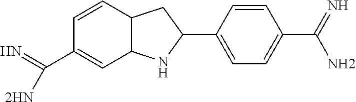 | DAPI | 343 | 455 |
| DAPI | | | |
| 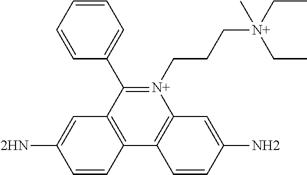 | Propidium | 536 | 617 |
| 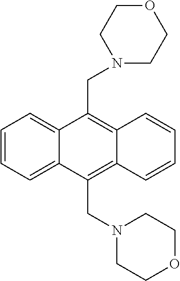 [SMILES: c1ccc2c(CN3CCOCC3)c3ccccc3c(CN3CCOCC3)c2c1] | Lysosensor Blue DND-167 | 373 | 425 |
| Lysosensor Blue DND-167 | | | |
| 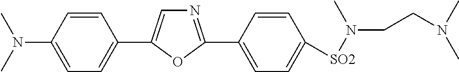 | Lysotracker Blue- White DPX | 373 | 430-640 |
| Lysotracker Blue-White DPX | | | |
| 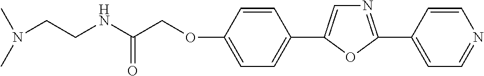 [SMILES: CN(C)CCNC(=O)COc1ccc(-c2cnc(-c3ccncc3)o2)cc1] | Lysosensor Yellow- DND-160 | 330 | 520 |
| Lysosensor Yellow/Blue DND-160 | | | |
| 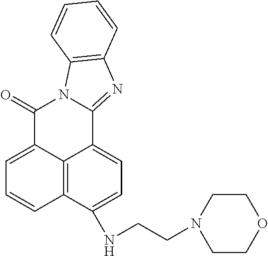 [SMILES: O=c1c2cccc3c(NCCN4CCOCC4)ccc(c32)c2nc3ccccc3n12] | Lysosensor Green, DND 189 | 443 | 505 |
| Lysosensor Green DND-189 | | | |
| 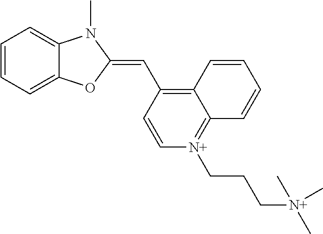 [SMILES: CN1/C(=C/c2cc[n+](CCC[N+](C)(C)C)c3ccccc23)Oc2ccccc21] | YO-PRO 1 | 491 | 503 |
| YO-PRO 1 491/631 | | | |
| 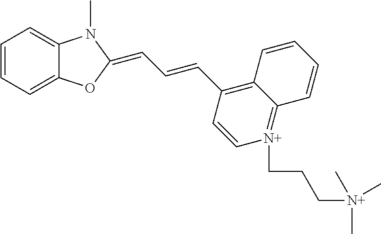 [SMILES: CN1/C(=C/C=C/c2cc[n+](CCC[N+](C)(C)C)c3ccccc23)Oc2ccccc21] | YO-PRO 3 | 612 | 631 |
| YO-PRO 3 612/631 | | | |
| 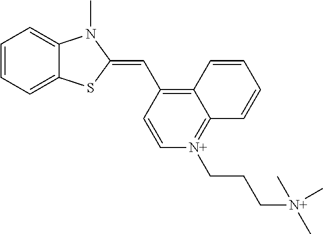 [SMILES: CN1/C(=C/c2cc[n+](CCC[N+](C)(C)C)c3ccccc23)Sc2ccccc21] | TO-PRO 1 | 515 | 531 |
| TO-PRO 1 515/531 | | | |
| 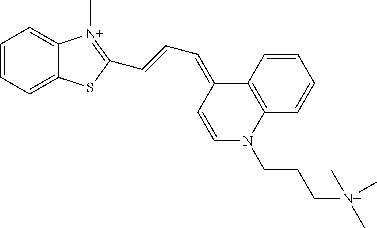 [SMILES: C[n+]1c(/C=C/C=C2\C=CN(CCC[N+](C)(C)C)c3ccccc32)sc2ccccc21] | TO-PRO 3 | 642 | 661 |
| TO-PRO 3 642/661 | | | |
| 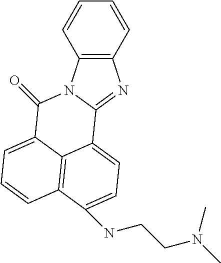 [SMILES: CN(C)CCNc1ccc2c3c1cccc3c(=O)n1c3ccccc3nc21] | Lysosensor Green DND-153 | 450 | 500 |
| 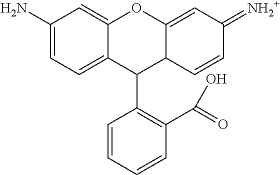 [SMILES: Nc1ccc2c(c1)OC1=CC(=[NH2+])C=CC1C2c1ccccc1C(=O)O] | Rhodamine 110 | 500 | 521 |
| Rhodamine 110 | | | |
| 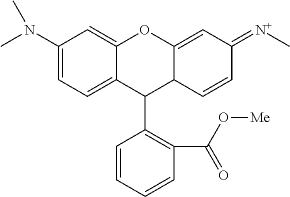 | TMR-Methylester | 550 | 575 |
| Tetramethyl Rhodamine Methyl Ester | | | |
| 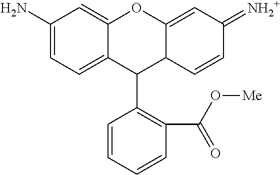 [SMILES: COC(=O)c1ccccc1C1c2ccc(N)cc2OC2=CC(=[NH2+])C=CC21] | Rhodamine 123 | 511 | 534 |
| Rhodamine 123 | | | |

TABLE 2
Commercially Available, Negatively Charged Alex Fluor Fluorochromes.

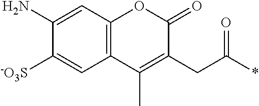

Alexa Fluor ® 360

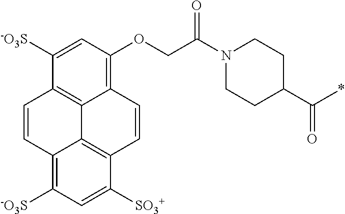

Alexa Fluor ® 405

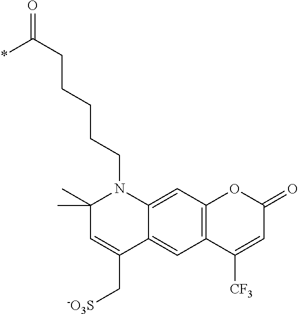

Alexa Fluor ® 430

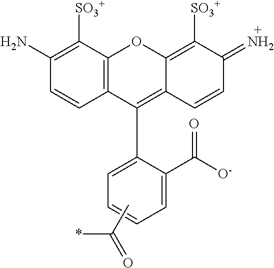

Alexa Fluor ® 488

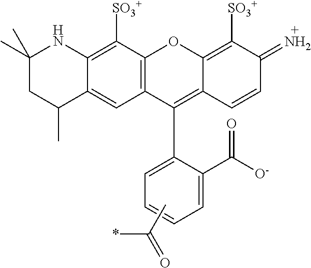

Alexa Fluor ® 514

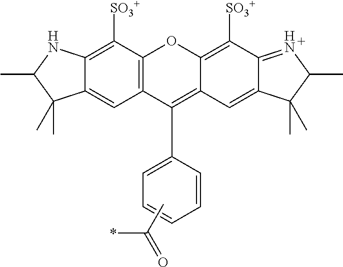

Alexa Fluor ® 532

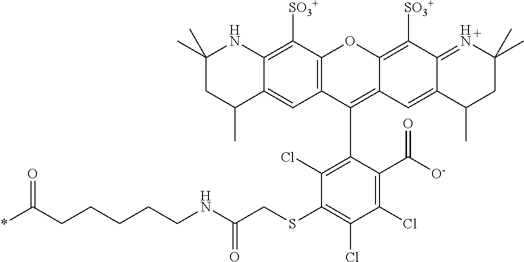

Alexa Fluor ® 546

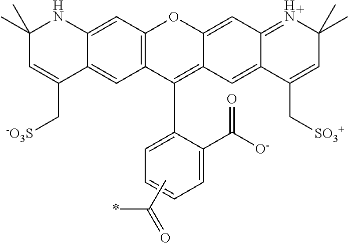

Alexa Fluor ® 568

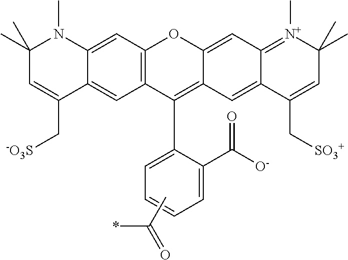

Alexa Fluor ® 594

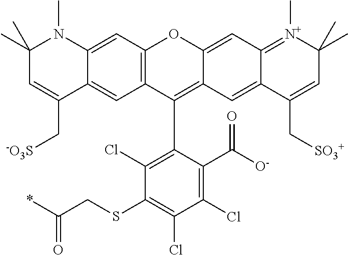

Alexa Fluor ® 610

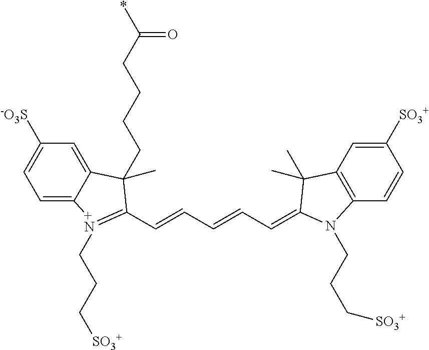

Alexa Fluor ® 647
The three number designations are the excitation maxima. The * indicates a linker and reactive group like an NHS ester

All references, patents, and patent publications described herein are incorporated by reference in total.

### REFERENCES

(1) Buxbaum, J. N., and Linke, R. P. (2012) A molecular history of the amyloidoses. Journal of molecular biology 421, 142-59.

(2) Chiti, F., and Dobson, C. M. (2006) Protein misfolding, functional amyloid, and human disease. Annual review of biochemistry 75, 333-66.

(3) Groenning, M. (2010) Binding mode of Thioflavin T and other molecular probes in the context of amyloid fibrils-current status. Journal of chemical biology 3, 1-18.

(4) Nelson, R., and Eisenberg, D. (2006) Structural models of amyloid-like fibrils. Advances in protein chemistry 73, 235-82.

(5) Zhuang, Z. P., Kung, M. P., Hou, C., Skovronsky, D. M., Gur, T. L., Plossl, K., Trojanowski, J. Q., Lee, V. M., and Kung, H. F. (2001) Radioiodinated styrylbenzenes and thioflavins as probes for amyloid aggregates. Journal of medicinal chemistry 44, 1905-14.

(6) Eckroat, T. J., Mayhoub, A. S., and Garneau-Tsodikova, S. (2013) Amyloid-beta probes: Review of structure-activity and brain-kinetics relationships. Beilstein journal of organic chemistry 9, 1012-44.

(7) Mathis, C. A., Mason, N. S., Lopresti, B. J., and Klunk, W. E. (2012) Development of positron emission tomography beta-amyloid plaque imaging agents. Seminars in nuclear medicine 42, 423-32.

(8) Necula, M., Kayed, R., Milton, S., and Glabe, C. G. (2007) Small molecule inhibitors of aggregation indicate that amyloid beta oligomerization and fibrillization pathways are independent and distinct. The Journal of biological chemistry 282, 10311-24.

(9) Qu, W., Choi, S. R., Hou, C., Zhuang, Z., Oya, S., Zhang, W., Kung, M. P., Manchandra, R., Skovronsky, D. M., and Kung, H. F. (2008) Synthesis and evaluation of indolinyl- and indolylphenylacetylenes as PET imaging agents for beta-amyloid plaques. Bioorganic & amp; medicinal chemistry letters 18, 4823-7.

(10) Hellstrom-Lindahl, E., Westermark, P., Antoni, G., and Estrada, S. (2014) In vitro binding of [(3)H]PIB to human amyloid deposits of different types. Amyloid: the international journal of experimental and clinical investigation: the official journal of the International Society of Amyloidosis 21, 21-7.

(11) Antoni, G., Lubberink, M., Estrada, S., Axelsson, J., Carlson, K., Lindsjo, L., Kero, T., Langstrom, B., Granstam, S. O., Rosengren, S., Vedin, O., Wassberg, C., Wikstrom, G., Westermark, P., and Sorensen, J. (2013) In vivo visualization of amyloid deposits in the heart with 11C-PIB and PET. Journal of nuclear medicine: official publication, Society of Nuclear Medicine 54, 213-20.

(12) Eberling, J. L., Dave, K. D., and Frasier, M. A. (2013) alpha-synuclein imaging: a critical need for Parkinson's disease research. Journal of Parkinson's disease 3, 565-7.

(13) Vernon, A. C., Ballard, C., and Modo, M. (2010) Neuroimaging for Lewy body disease: is the in vivo molecular imaging of alpha-synuclein neuropathology required and feasible? Brain research reviews 65, 28-55.

(14) Luhrs, T., Ritter, C., Adrian, M., Riek-Loher, D., Bohrmann, B., Dobeli, H., Schubert, D., and Riek, R. (2005) 3D structure of Alzheimer's amyloid-beta(1-42) fibrils. Proceedings of the National Academy of Sciences of the United States of America 102, 17342-7.

(15) Wacker, J. L., Zareie, M. H., Fong, H., Sarikaya, M., and Muchowski, P. J. (2004) Hsp70 and Hsp40 attenuate formation of spherical and annular polyglutamine oligomers by partitioning monomer. Nature structural & amp; molecular biology 11, 1215-22.

(16) Celej, M. S., Sarroukh, R., Goormaghtigh, E., Fidelio, G. D., Ruysschaert, J. M., and Raussens, V. (2012) Toxic prefibrillar alpha-synuclein amyloid oligomers adopt a distinctive antiparallel beta-sheet structure. The Biochemical journal 443, 719-26.

(17) Westermark, P., Andersson, A., and Westermark, G. T. (2011) Islet amyloid polypeptide, islet amyloid, and diabetes mellitus. Physiological reviews 91, 795-826.

(18) DeToma, A. S., Salamekh, S., Ramamoorthy, A., and Lim, M. H. (2012) Misfolded proteins in Alzheimer's disease and type II diabetes. Chem Soc Rev 41, 608-21.

(19) Mukrasch, M. D., Bibow, S., Korukottu, J., Jeganathan, S., Biernat, J., Griesinger, C., Mandelkow, E., and Zweckstetter, M. (2009) Structural polymorphism of 441-residue tau at single residue resolution. PLoS biology 7, e34.

(20) Ross, C. A., and Tabrizi, S. J. (2011) Huntington's disease: from molecular pathogenesis to clinical treatment. The Lancet. Neurology 10, 83-98.

(21) Poirier, M. A., Jiang, H., and Ross, C. A. (2005) A structure-based analysis of huntingtin mutant polyglutamine aggregation and toxicity: evidence for a compact beta-sheet structure. Hum Mol Genet 14, 765-74.

(22) Goedert, M. (2001) Alpha-synuclein and neurodegenerative diseases. Nat Rev Neurosci 2, 492-501.

(23) Shammas, S. L., Garcia, G. A., Kumar, S., Kjaergaard, M., Horrocks, M. H., Shivji, N., Mandelkow, E., Knowles, T. P., Mandelkow, E., and Klenerman, D. (2015) A mechanistic model of tau amyloid aggregation based on direct observation of oligomers. Nature communications 6, 7025.

(24) Alderson, T. R., and Markley, J. L. (2013) Biophysical characterization of alpha-synuclein and its controversial structure. Intrinsically disordered proteins 1, 18-39.

(25) Bertoncini, C. W., Jung, Y. S., Fernandez, C. O., Hoyer, W., Griesinger, C., Jovin, T. M., and Zweckstetter, M. (2005) Release of long-range tertiary interactions potentiates aggregation of natively unstructured alpha-synuclein. Proceedings of the National Academy of Sciences of the United States of America 102, 1430-5.

(26) Vilar, M., Chou, H. T., Luhrs, T., Maji, S. K., Riek-Loher, D., Verel, R., Manning, G., Stahlberg, H., and Riek, R. (2008) The fold of alpha-synuclein fibrils. Proceedings of the National Academy of Sciences of the United States of America 105, 8637-42.

(27) Rodriguez-Rodriguez, C., Rimola, A., Rodriguez-Santiago, L., Ugliengo, P., Alvarez-Larena, A., Gutierrez-de-Teran, H., Sodupe, M., and Gonzalez-Duarte, P. (2010) Crystal structure of thioflavin-T and its binding to amyloid fibrils: insights at the molecular level. Chemical communications (Cambridge, England) 46, 1156-8.

(28) Noel, S., Cadet, S., Gras, E., and Hureau, C. (2013) The benzazole scaffold: a SWAT to combat Alzheimer's disease. Chemical Society Reviews 42, 7747-7762.

(29) Goedert, M. (2001) Parkinson's disease and other alpha-synucleinopathies. Clinical chemistry and laboratory medicine 39, 308-12.

(30) Boens, N., Leen, V., and Dehaen, W. (2012) Fluorescent indicators based on BODIPY. Chem Soc Rev 41, 1130-72.

(31) Leen, V., Braeken, E., Luckermans, K., Jackers, C., Van der Auweraer, M., Boens, N., and Dehaen, W. (2009) A versatile, modular synthesis of monofunctionalized BODIPY dyes. Chemical communications (Cambridge, England), 4515-7.

(32) Glazer, A. N., and Rye, H. S. (1992) Stable dye-DNA intercalation complexes as reagents for high-sensitivity fluorescence detection. Nature 359, 859-61.

(33) Fei, X., Gu, Y., Ban, Y., Liu, Z., and Zhang, B. (2009) Thiazole Orange derivatives: synthesis, fluorescence properties, and labeling cancer cells. Bioorganic & amp; medicinal chemistry 17, 585-91.

(34) Fei, X., Yang, S., Zhang, B., Liu, Z., and Gu, Y. (2007) Solid-phase synthesis and modification of Thiazole Orange and its derivatives and their spectral properties. J Comb Chem 9, 943-50.

(35) Choi, H. S., Gibbs, S. L., Lee, J. H., Kim, S. H., Ashitate, Y., Liu, F., Hyun, H., Park, G., Xie, Y., Bae, S., Henary, M., and Frangioni, J. V. (2013) Targeted zwitterionic near-infrared fluorophores for improved optical imaging. Nature biotechnology 31, 148-53.

(36) Choi, H. S., Nasr, K., Alyabyev, S., Feith, D., Lee, J. H., Kim, S. H., Ashitate, Y., Hyun, H., Patonay, G., Strekowski, L., Henary, M., and Frangioni, J. V. (2011) Synthesis and in vivo fate of zwitterionic near-infrared fluorophores. Angewandte Chemie (International ed. in English) 50, 6258-63.

(37) Lin, Y., Weissleder, R., and Tung, C. H. (2002) Novel near-infrared cyanine fluorochromes: synthesis, properties, and bioconjugation. Bioconjug Chem 13, 605-10.

(38) Shi, C., Wu, J. B., and Pan, D. (2016) Review on near-infrared heptamethine cyanine dyes as theranostic agents for tumor imaging, targeting, and photodynamic therapy. J Biomed Opt 21, 50901.

(39) Yang, X., Shi, C., Tong, R., Qian, W., Zhau, H. E., Wang, R., Zhu, G., Cheng, J., Yang, V. W., Cheng, T., Henary, M., Strekowski, L., and Chung, L. W. (2010) Near IR heptamethine cyanine dye-mediated cancer imaging. Clinical cancer research: an official journal of the American Association for Cancer Research 16, 2833-44.

I claim:

1. A method for identifying an interaction between a peptide bearing a partial amino acid sequence of a known protein and a fluorochrome, comprising:
(a) contacting a first peptide and a first fluorochrome, and measuring the fluorescence emitted from the first peptide-first fluorochrome pair;
(b) contacting a second peptide with the first fluorochrome, wherein the second peptide bears the same amino acids as the first peptide in a scrambled order; and measuring the fluorescence emitted from the second peptide-first fluorochrome pair;
(c) comparing the fluorescence emitted from the first peptide-first fluorochrome pair with the fluorescence emitted from second peptide-first fluorochrome pair, wherein a difference in fluorescence emitted between the first peptide-first fluorochrome pair and the second peptide-first fluorochrome pair indicates a sequence dependent interaction.

2. The methods of claim 1 wherein the difference in fluorescence emitted is an increase in fluorescence of the first fluorochrome pair over the second peptide-first fluorochrome pair.

3. The method of claim 1 wherein the difference in fluorescence emitted is decrease in fluorescence of the first fluorochrome pair over the second peptide-first fluorochrome pair.

4. The method of claim 2 wherein the first peptide is located in the non-hairpin beta sheet portion of an amyloid forming protein and the second peptide is a scrambled amino acid sequence of the first peptide.

5. The method of claim 2 where in the first peptide is located in the non-hairpin beta-sheet portion of ASyn.

6. The method of claim 2 where in the first peptide is Seq. ID. No. 1

6. The method of claim 2 wherein the first peptide is Seq. ID No. 1 and the second peptide is Seq. ID. No. 2.

7. The method of claim of 2 wherein the first peptide is located in the non-hairpin beta-sheet portion of Tau.

8. The method of claim 2 where in the first peptide is Seq. Id. No. 3.

9. A method for detecting an interaction of a test compound with a partial amino acid sequence of a known protein, comprising,
(a) obtaining the first peptide-first fluorochrome pair of claim 1 and measuring the fluorescence emitted by the pair;
(b) forming a mixture by combining (i) a non-fluorescent test compound, and (ii) the first peptide-first fluorochrome pair; and
(b) measuring the fluorescence emitted by the fluorochrome in the presence of the test compound, wherein a decrease of fluorescence in the mixture compared to the fluorescence in step (a) indicates an interaction between the test compound and the first peptide.

10. The method of claim 9 wherein the first peptide is located in the non-hairpin beta sheet portion of an amyloid forming protein.


SEQUENCE LISTING (5 sequences)

SEQ ID NO: 1 - protein, 42 aa, Homo sapiens [sequence omitted]

SEQ ID NO: 2 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 3 - protein, 50 aa, Homo sapiens
MDVFMKGLSKAKEGVVAAAEKTKQGVAEAAGKTKEGVLYVGSKTKEGVVH

SEQ ID NO: 4 - protein, 37 aa, Homo sapiens
KCNTATCARQRLANFLVHSSNNFGAILSSTNVGSNTY

SEQ ID NO: 5 - protein, 415 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSST